(19) United States
(12) Patent Application Publication
(10) Pub. No.: US 2024/0215872 A1
(43) Pub. Date: Jul. 4, 2024

(54) CURVED NON-INVASIVE GLUCOSE MONITORING MODULE

(71) Applicant: Taiwan-Asia Semiconductor Corporation, Hsinchu City (TW)

(72) Inventors: Hsiu-Jung HUANG, Hsinchu City (TW); Sheng-Wei CHEN, Hsinchu City (TW)

(21) Appl. No.: 18/515,642

(22) Filed: Nov. 21, 2023

(30) Foreign Application Priority Data
Dec. 29, 2022 (TW) 111150768

(51) Int. Cl. A61B 5/145; A61B 5/1455

(52) U.S. Cl. CPC A61B 5/14532; A61B 5/1455; A61B 2560/0406; A61B 2560/0462; A61B 2562/0238; A61B 2562/16

(57) ABSTRACT

The invention provides a curved non-invasive glucose monitoring module includes a substrate, a barrier structure, a light emitting element, a monitoring element and a transparent cover. The barrier structure, the light-emitting element and the monitoring element are all arranged on the substrate. The barrier structure forms a first space and a second space isolated from each other. The light emitting element is located in the first space, and the monitoring element is located in the second space. The transparent cover is combined with the barrier structure to close the first space and the second space. At least one of the substrate and the transparent cover is a curved structure.

### CROSS REFERENCE TO RELATED APPLICATION

This application claims the benefit of Taiwan Patent Application Serial No. 111150768 filed on Dec. 29, 2022. The entirety of each Application is incorporated herein by reference.

### BACKGROUND OF THE INVENTION

### 1. Field of the Invention

The present invention relates to a blood glucose monitoring module, especially a non-invasive blood glucose monitoring module with a curved structure.

### 2. Description of Related Art

Common blood glucose monitoring modules on the market can be roughly divided into invasive blood glucose monitoring modules and non-invasive blood glucose monitoring modules. The non-invasive blood glucose monitoring module mainly uses a sensing surface to approach or contact human skin, and then drives a light emitting element to emit light in specific wavelengths. The light passes through a cover of the module, and reaches the tissues under the skin for generating a diffuse reflection. Finally, the reflected light is received by a sensing element to detect related values of blood glucose.

However, conventional non-invasive blood glucose monitoring modules adopt designs of flat covers. When the conventional non-invasive blood glucose monitoring module approaches or contacts the skin, uneven height differences will form between the flat cover and the skin, and thus causes that the light is unable to reach the skin successfully, which is likely to induce situations of sensing errors and even of sensing failures. In addition, when the conventional non-invasive blood glucose monitoring module is combined with a device with a streamlined appearance (such as sporting equipment, glasses or smart wearable devices) for monitoring, the design of the flat cover ruins the streamlined appearance of the device and also tends to bring a bad user-experience for the device.

Therefore, how to design a non-invasive blood glucose monitoring module that can improve the aforementioned issues is indeed a research-worthy topic.

### SUMMARY OF THE INVENTION

An objective of the present invention is to provide a non-invasive blood glucose monitoring module with a curved structure.

To achieve the above mentioned objective, the curved non-invasive blood glucose monitoring module of the present invention comprises a substrate, a barrier structure, a light emitting element, a sensing element and a transparent cover. The barrier structure, the light emitting element and the sensing element are disposed on the substrate. The barrier structure forms a first space and a second space isolated from each other. The light emitting element is located in the first space and the sensing element is located in the second space. The transparent cover is combined with the barrier structure to enclose the first space and the second space. Wherein at least one of the substrate and the transparent cover is a curved structure.

In one embodiment of the present invention, the barrier structure includes a main retaining wall and a peripheral retaining wall, and wherein the main retaining wall is disposed between the first space and the second space, and the peripheral retaining wall connects the main retaining wall and surrounds the main retaining wall, the first space and the second space.

In one embodiment of the present invention, the transparent cover is a convex cover, the substrate is a flat substrate, and a height of the main retaining wall is greater than a height of the peripheral retaining wall.

In one embodiment of the present invention, the transparent cover is a convex cover, the substrate is a convex substrate, and a height of the main retaining wall equals to a height of the peripheral retaining wall.

In one embodiment of the present invention, the transparent cover is a concave cover, the substrate is a flat substrate, and a height of the main retaining wall is less than a height of the peripheral retaining wall.

In one embodiment of the present invention, the transparent cover is a flat cover, the substrate is a convex substrate, and a height of the main retaining wall is less than a height of the peripheral retaining wall.

In one embodiment of the present invention, the barrier structure is made of non-transparent materials.

In one embodiment of the present invention, a transparent encapsulant material is filled within the first space and the second space to protect the light emitting element and the sensing element.

In one embodiment of the present invention, a radius of curvature of the curved structure is between 10 mm and 1000 mm.

In one embodiment of the present invention, the transparent cover includes a light emitting coating film and a light receiving coating film, and wherein a configured position of the light emitting coating film corresponds to the position of the first space, and a configured position of the light receiving coating film corresponds to the position of the second space.

Hereby, when the curved non-invasive blood glucose monitoring module according to the present invention is attached on the target device or the component, through the design of the curved structure of the substrate and/or the transparent cover, it well matches the original appearance of the target device or the component and thus uneven height differences won't unnecessarily occur to influence sensing accuracy or effect. Furthermore, the curved structure adopted by the module is beneficial to keeping the streamlined appearance of the target device or the component, or beneficial to installing on the target device or the component with the streamlined appearance.

### BRIEF DESCRIPTION OF THE DRAWINGS

FIG. 1 is a schematic view of a first embodiment of a curved non-invasive blood glucose monitoring module according to the present invention;

FIG. 2 is a schematic view of a second embodiment of the curved non-invasive blood glucose monitoring module according to the present invention;

FIG. 3 is a schematic view of a third embodiment of the curved non-invasive blood glucose monitoring module according to the present invention; and

FIG. 4 is a schematic view of a fourth embodiment of the curved non-invasive blood glucose monitoring module according to the present invention.

### DETAILED DESCRIPTION OF THE PREFERRED EMBODIMENT

Since various examples and embodiments in the present invention are only illustrative and non-restrictive, a person skilled in the art can easily conceive other examples and embodiments without contravening the scope of the present invention, after reading this specification, and can make the features and advantages of these embodiments more evident based on the following detailed description and claims.

Herein, the description of unit, element and component in the present invention uses “one”, “a”, or “an”. This is for convenience and for offering general meaning of the category of the present invention. Therefore, the description should be understood as including “one”, “at least one”, and singular and plural forms at the same time unless the context clearly indicates otherwise.

Herein, the description of the terms “first” or “second” and similar ordinal numbers are mainly used to distinguish or refer to the same or similar elements or structures and do not necessarily imply that such components or structures are spatially or temporally distinct order. It should be understood that ordinal numbers, in certain situations or configurations, may be used interchangeably without affecting the implementation of the present invention.

Herein, the description of “comprise”, “have” or other similar semantics have the non-exclusive meaning. For example, components or structures with a plurality of elements are not only limited to those disclosed in this specification, but also include generally inherent elements, which are not explicitly listed here for the components or the structures.

Herein, the term “convex structure” is defined as a curved structure that bulges toward the front of the module (i.e., facing toward subjects, to be detected, such as skins) and exhibits a curvature, while the term “concave structure” is defined as a curved structure that depresses toward the rear of the module (i.e., facing away from subjects, to be detected, such as skins) and exhibits a curvature.

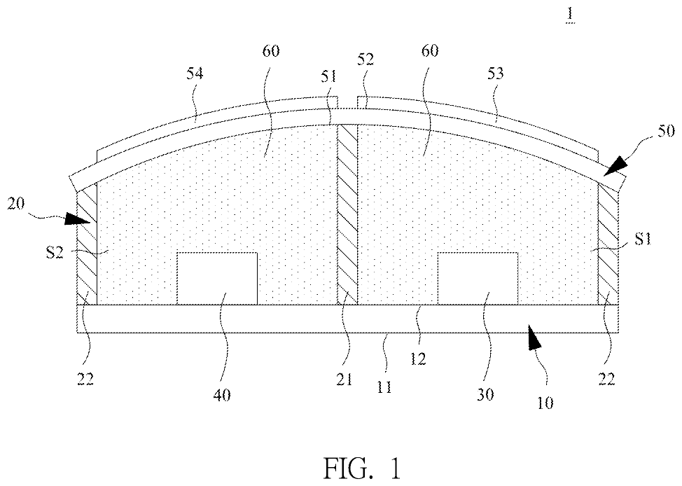

Please refer to FIG. 1, which is a schematic diagram of a first embodiment of a curved non-invasive blood glucose monitoring module of the present invention. As shown in FIG. 1, the curved non-invasive blood glucose monitoring module 1 of the present invention principally comprises a substrate 10, a barrier structure 20, a light emitting element 30, a sensing element 40 and a transparent cover 50. The substrate 10 is mainly served as a basic structure of the curved non-invasive blood glucose monitoring module 1, and the substrate 10 is adopted to carry the barrier structure 20, the light emitting element 30, the sensing element 40 and the transparent cover 50, thus forming an integral module. The substrate 10 includes an installing surface 11 and a carrying surface 12 opposite to each other. The installing surface 11 is used to attach on a target device or a component that needs to provide a blood glucose monitoring function, while the carrying surface 12 is used to carry the main components of the aforementioned module. In the present invention, according to different design requirements, the substrate 10 can be a flat structure or a curved structure, and in this embodiment, the substrate 10 is a flat substrate. Selectively, the substrate 10 can adopt from a ceramic substrate, a silicon substrate or a PCB substrate, but not limited thereto.

The barrier structure 20 is disposed on the carrying surface 12 of the substrate 10, and the barrier structure 20 forms a first space S1 and a second space S2 isolated from each other. In one embodiment of the present invention, the barrier structure 20 includes a main retaining wall 21 and a peripheral retaining wall 22. The main retaining wall 21 is disposed between the first space S1 and the second space S2, and the peripheral retaining wall 22 connects the main retaining wall 21 and surrounds the main retaining wall 21, the first space S1 and the second space S2. The barrier structure 20 is made of non-transparent (including visible light and invisible light) materials, such as black glue material or other lightproof glue materials. Accordingly, the barrier structure 20 can prevent mutual influencing between the first space S1 serving as a light emitting side and the second space S2 serving as a light receiving side. In the present invention, the main retaining wall 21 and the peripheral retaining wall 22 may have equal or different heights according to different design requirements, and in this embodiment, a height of the main retaining wall 21 is greater than a height of the peripheral retaining wall 22.

The light emitting element 30 is disposed on the carrying surface 12 of the substrate 10, and the light emitting element 30 is located in the first space S1. The light emitting element 30 mainly emits light toward skins. In the present invention, a single LED light source is used for the light emitting element 30, but the type, location and quantity of the light emitting element 30 can be adjusted according to different design requirements. For instance, the aforementioned light emitting element 30 may adopt an LED light source for emitting short wave infrared (SWIR), with a spectral range between 700 nm and 2000 nm, but not limited thereto. The light emitting element 30 can be electrically connected to the substrate 10 through solder wires (not shown in FIG. 1).

The sensing element 40 is disposed on the carrying surface 12 of the substrate 10, and the sensing element 40 is located in the second space S2. The sensing element 40 mainly receives light back from the skins by diffuse reflection. In the present invention, one photo detector is used for the sensing element 40, but the type, location and quantity of the sensing element 40 can be adjusted according to different design requirements. For example, the sensing element 40 may adopt a photo detector made of indium gallium arsenide (InGaAs), with a light absorption range between 900 nm and 1700 nm, but not limited thereto. The sensing element 40 can also be electrically connected to the substrate 10 through solder wires (not shown in FIG. 1).

The transparent cover 50 is configured with the barrier structure 20, and openings of the first space SI and the second space S2 are enclosed by bonding of the transparent cover 50 and the barrier structure 20, so that the first space S1 and the second space S2 respectively form enclosed spaces independent from each other. The transparent cover 50 includes a first surface 51 and a second surface 52 opposite to each other. The first surface 51 is used to connect to the barrier structure 20, and the second surface 52 is used to approach or contact subjects, to be detected, such as skins. In the present invention, according to different design requirements, the transparent cover 50 can be a flat structure or a curved structure, and in this embodiment, the transparent cover 50 is a convex cover. A radius of curvature of the aforementioned curved structure (the convex cover) is between 10 mm and 1000 mm.

The transparent cover 50 is made of transparent materials, such as glass, but not limited thereto. The transparent cover 50 further includes a light emitting coating film 53 and a light receiving coating film 54. Wherein the light emitting coating film 53 and the light receiving coating film 54 are both formed on second surface 52 of the transparent cover 50, and wherein a configured position of the light emitting coating film 53 corresponds to the position of the first space S1, and a configured position of the light receiving coating film 54 corresponds to the position of the second space S2. The structures and functional applications of the light emitting coating film 53 and the light receiving coating film 54 can be adjusted according to different design requirements. Since the light emitting coating film 53 and the light receiving coating film 54 are structural designs commonly used in conventional covers, no further details are given here. For instance, a dual band pass filter film, which is mainly a complex layered structure formed by alternately stacking of silicon monohydride (SiH) layers and silicon dioxide (SiO2) layers, is used for the aforementioned light emitting coating film 53 and the light receiving coating film 54, but not limited thereto in the present invention. By disposing of the light emitting coating film 53 and the light receiving coating film 54, the transparent cover 50 can provide an effect of allowing light in a specific wavelength range to pass through, and can effectively block stray light in other unnecessary wavelength ranges.

In the present invention, the transparent cover 50 is bonded to the barrier structure 20 by gluing. For example, the first space S1 and the second space S2 of the curved non-invasive blood glucose monitoring module 1 of the present invention can be filled with a transparent encapsulant material 60, such as a liquid optical glue or an epoxy, but not limited thereto. Due to adhesive force of the transparent encapsulant material 60, as the transparent cover 50 is in contact with the barrier structure 20, the transparent encapsulant material 60 provides an adhesive fixing effect on the transparent cover 50, so as to enhance bonding of the transparent cover 50 and the barrier structure 20. On the other hand, the transparent sealing material 60 can also provide a packaging effect for the light emitting element 30, the sensing element 40 and the aforementioned solder wires and provide an effect of optical transmission property.

The first embodiment of the curved non-invasive blood glucose monitoring module 1 of the present invention mainly adopts a convex cover for the transparent cover 50 and a flat substrate for the substrate 10. In order to cooperate with the design of the aforementioned substrate 10 and transparent cover 50, a height of the main retaining wall 21 will be greater than a height of the peripheral retaining wall 22, to facilitate bonding of the transparent cover 50 with the barrier structure 20 for strictly sealing the first space S1 and the second space S2. Accordingly, the curved non-invasive blood glucose monitoring module 1 of the present invention can be attached on an exterior surface or into a flat-bottomed groove of the target device or the component (such as a handle of sporting equipment) through the substrate 10, and maintains the consistency of overall appearance by that the transparent cover 50 can match a streamlined appearance of the target device or the component. In addition, since the transparent cover 50 is a curved structure, when a user holds the handle with his hand, the skin of his hand can completely attach to the second surface 52 of the transparent cover 50, and an unevenly attaching situation (a distance therebetween is consistent or zero) is unlikely to occur. Thereby, a possibility of poor light transmission, due to a gap existing between the second surface 52 of the transparent cover 50 and the skin of the hand, is reduced.

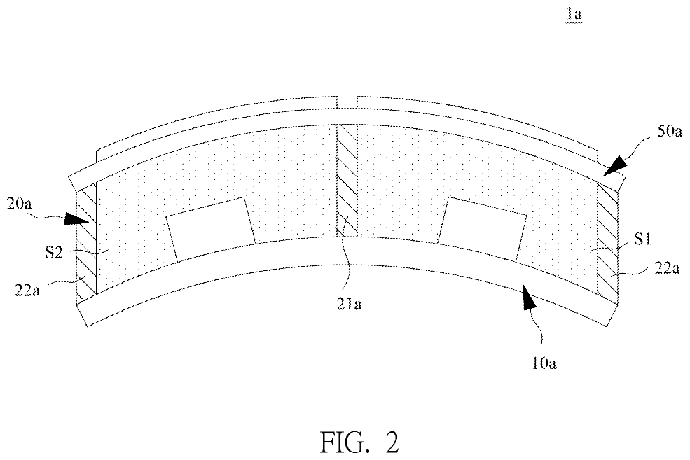

Please refer to FIG. 2, which is a schematic view of a second embodiment of the curved non-invasive blood glucose monitoring module according to the present invention. This embodiment is a modification of the aforementioned first embodiment, mainly by modifying the structural type of the substrate. As shown in FIG. 2, the second embodiment of the curved non-invasive blood glucose monitoring module la of the present invention mainly adopts a convex cover for the transparent cover 50a and a convex substrate for the substrate 10a. In order to cooperate with the design of the aforementioned substrate 10a and transparent cover 50a, the height of the main retaining wall 21a will be equal to the height of the peripheral retaining wall 22a, so as to facilitate bonding of the transparent cover 50a with the barrier structure 20a for strictly sealing the first space S1 and the second space S2. Hereby, the curved non-invasive blood glucose monitoring module la of the present invention can be attached on an exterior curved surface or into a convex-bottomed groove of the target device or the component (such as the handle of sporting equipment) through the substrate 10a, and maintains the consistency of overall appearance by that the transparent cover 50a can still match the streamlined appearance of the target device or the component. In addition, this embodiment also provides the same effect of the aforementioned first embodiment.

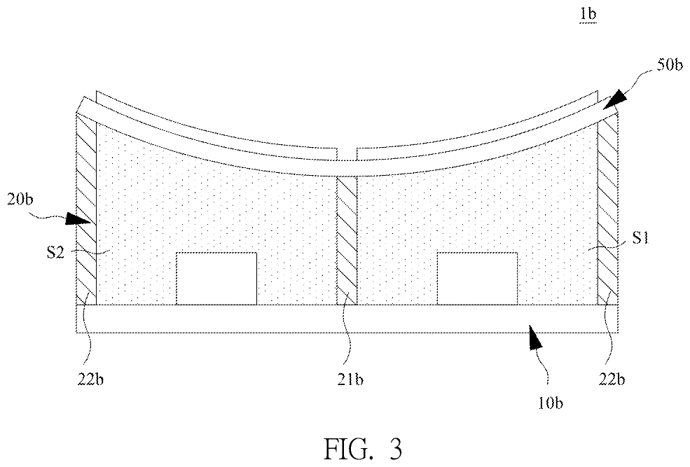

Please refer to FIG. 3, which is a schematic view of a third embodiment of the curved non-invasive blood glucose monitoring module according to the present invention. This embodiment is a modification of the aforementioned first embodiment, mainly by modifying the structural type of the transparent cover. As shown in FIG. 3, the third embodiment of the curved non-invasive blood glucose monitoring module 1b of the present invention mainly adopts a concave cover for the transparent cover 50b and a flat substrate for the substrate 10b. In order to cooperate with the design of the aforementioned substrate 10b and transparent cover 50b, the height of the main retaining wall 21b will be less than the height of the peripheral retaining wall 22b, so as to facilitate bonding of the transparent cover 50b with the barrier structure 20b for strictly sealing the first space S1 and the second space S2. Accordingly, the curved non-invasive blood glucose monitoring module 1b of the present invention can be attached on an exterior surface or into a flat-bottomed groove of the target device or the component (such as a hollow sheath or a concave structural design for accommodating fingertips of sporting equipment) through the substrate 10b, and maintains the consistency of overall appearance by that the transparent cover 50b can still match the streamlined appearance of the target device or the component. Moreover, this embodiment also provides the same effect of the aforementioned first embodiment.

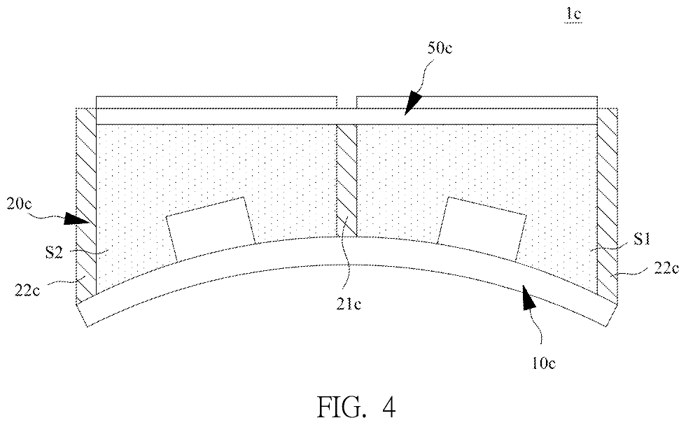

Please refer to FIG. 4, which is a schematic view of a fourth embodiment of the curved non-invasive blood glucose monitoring module according to the present invention. This embodiment is a modification of the aforementioned second embodiment, mainly by modifying the structural type of the transparent cover. As shown in FIG. 4, the fourth embodiment of the curved non-invasive blood glucose monitoring module 1c of the present invention mainly adopts a flat cover for the transparent cover 50c and a convex substrate for the substrate 10c. In order to cooperate with the design of the aforementioned substrate 10c and transparent cover 50c, the height of the main retaining wall 21c will be less than the height of the peripheral retaining wall 22c, so as to facilitate bonding of the transparent cover 50c with the barrier structure 20c for strictly sealing the first space S1 and the second space S2. Hereby, the curved non-invasive blood glucose monitoring module 1c of the present invention can be attached on an exterior curved surface or into a convex-bottomed groove of the target device or component through the substrate 10c, and maintains the consistency of overall appearance by that the transparent cover 50c can match the streamlined appearance of the target device or the component.

Accordingly, the curved non-invasive blood glucose monitoring module according to the present invention adopts the curved structure for at least one of the substrate and the transparent cover, so that it can be completely attached to skins of users for improving the monitoring effect and/or for easy attaching on the target device or the component with the streamlined appearance, which improves the convenience of use and/or the flexibility of arrangement of the curved non-invasive blood glucose monitoring module according to the present invention.

The above implementations are only auxiliary descriptions, and are not intended to limit the embodiments of the application subject or the applications or uses of the embodiments. In addition, although at least one illustrative example has been presented above, it should be understood that the present invention can still have a large number of variations. It should also be understood that the embodiments described herein are not intended to limit the scope, use, or configuration of the requested subject matter in any way. On the contrary, the foregoing embodiments will provide a convenient guide for those skilled in the art to implement one or more embodiments. Furthermore, various changes can be made to the function and arrangement of the components without departing from the scope defined by the patent claims, and the scope of the patent claims includes known equivalents and all foreseeable equivalents at the time that the patent application is filed.

What is claimed is:

1. A curved non-invasive blood glucose monitoring module, comprising:
a substrate,
a barrier structure, disposed on the substrate and forming a first space and a second space isolated from each other,
a light emitting element, disposed on the substrate and located in the first space,
a sensing element, disposed on the substrate and located in the second space, and
a transparent cover, combined with the barrier structure to enclose the first space and the second space,
wherein at least one of the substrate and the transparent cover is a curved structure.

2. The curved non-invasive blood glucose monitoring module as claimed in claim 1, wherein the barrier structure includes a main retaining wall and a peripheral retaining wall, and wherein the main retaining wall is disposed between the first space and the second space, and the peripheral retaining wall connects the main retaining wall and surrounds the main retaining wall, the first space and the second space.

3. The curved non-invasive blood glucose monitoring module as claimed in claim 2, wherein the transparent cover is a convex cover, the substrate is a flat substrate, and a height of the main retaining wall is greater than a height of the peripheral retaining wall.

4. The curved non-invasive blood glucose monitoring module as claimed in claim 2, wherein the transparent cover is a convex cover, the substrate is a convex substrate, and a height of the main retaining wall equals to a height of the peripheral retaining wall.

5. The curved non-invasive blood glucose monitoring module as claimed in claim 2, wherein the transparent cover is a concave cover, the substrate is a flat substrate, and a height of the main retaining wall is less than a height of the peripheral retaining wall.

6. The curved non-invasive blood glucose monitoring module as claimed in claim 2, wherein the transparent cover is a flat cover, the substrate is a convex substrate, and a height of the main retaining wall is less than a height of the peripheral retaining wall.

7. The curved non-invasive blood glucose monitoring module as claimed in claim 1, wherein the barrier structure is made of non-transparent materials.

8. The curved non-invasive blood glucose monitoring module as claimed in claim 1, wherein a transparent encapsulant material is filled within the first space and the second space to protect the light emitting element and the sensing element.

9. The curved non-invasive blood glucose monitoring module as claimed in claim 1, wherein a radius of curvature of the curved structure is between 10 mm and 1000 mm.

10. The curved non-invasive blood glucose monitoring module as claimed in claim 1, wherein the transparent cover includes a light emitting coating film and a light receiving coating film, and wherein a configured position of the light emitting coating film corresponds to the position of the first space, and a configured position of the light receiving coating film corresponds to the position of the second space.
